SEQUENCE LISTING (3 sequences)

SEQ ID NO: 1 - protein, 431 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 552 aa, Artificial Sequence
  Conbercept
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRLPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 3 - protein, 126 aa, Artificial Sequence
  DARPin MP0112
GSDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAA

(19) United States
(12) Patent Application Publication
(10) Pub. No.: US 2016/0137717 A1
(43) Pub. Date: May 19, 2016

(54) USE OF A VEGF ANTAGONIST IN TREATING CHOROIDAL NEOVASCULARISATION

(71) Applicant: Gabriela BURIAN, Basel (CH)

(72) Inventor: Gabriela BURIAN, Basel (CH)

(21) Appl. No.: 14/898,294

(22) PCT Filed: Jun. 18, 2014
(86) PCT No.: PCT/IB2014/062371

§ 371 Date: Dec. 14, 2015

Related U.S. Application Data
(60) Provisional application No. 61/837,250, filed on Jun. 20, 2013.

(51) Int. Cl. C07K 14/71; A61K 38/17; A61K 45/06

(52) U.S. Cl. CPC C07K 14/71; A61K 45/06; A61K 38/179; C07K 2319/30

(57) ABSTRACT

The present invention relates to the use of a non-antibody VEGF antagonist, in the treatment of choroidal neovascularisation secondary to diseases other than age-related macular degeneration and pathologic myopia.

### TECHNICAL FIELD

This invention is in the field of treating retinal disorders. In particular, the present invention relates to the treatment of choroidal neovascularisation secondary to diseases other than age-related macular degeneration and pathologic myopia.

### BACKGROUND ART

The growth of new blood vessels that originate from the choroid (the vascular layer of the eye between the retina and the sclera) and enter the sub-retinal pigment epithelium or subretinal space is referred to as “choroidal neovascularisation” (CNV). The most common causes of CNV are age-related macular degeneration and pathologic myopia. VEGF is a well-characterised signal protein which stimulates angiogenesis and is directly involved in neovascularisation. VEGF antagonists such as pegaptanib (Macugen®), ranibizumab (Lucentis®) and bevacizumab (Avastin®) have been successfully used for treating CNV in patients suffering from CNV secondary to age-related macular degeneration and pathologic myopia.

CNV is often associated with inflammation Inflammatory processes can be triggered by an infectious agent such as a fungus, roundworm or protozoan. In other cases, an autoimmune response may be the underlying cause of inflammation. Examples for inflammatory CNV include CNV secondary to uveitis, toxoplasmosis, toxocariasis, multifocal chorioditis, (presumed) ocular histoplasmosis, punctate inner choroidopathy, scleroderma, serpiginous choriodopathy, and Vogt-Koyanagi-Harada syndrome.

In many cases, the underlying mechanism causing CNV is unknown. These cases are referred to as idiopathic CNV. For example, CNV is observed in patients having angioid streaks or central serous chorioretinopathy, and there is not always an obvious link to an underlying disease or disorder. CNV can also be associated with rare benign tumours such as choroidal osteoma or certain genetic diseases such as pseudoxanthoma elasticum, Paget's disease, sickle cell anemia and Ehlers-Danlos Syndrome. These diseases may be associated with the formation of angioid streaks, with CNV usually developing secondarily to angioid streaks.

For many years, the standard treatments for CNV were limited to laser photocoagulation therapy (LPT), photodynamic therapy (PDT) and submacular surgery. However, these treatments sometimes can themselves cause (iatrogenic) CNV due to the damage caused by the laser treatment and the subsequent healing process. In recent years, off-label use of ranibizumab (Lucentis®) and bevacizumab (Avastin®) has shown promising results in treating CNV secondary to diseases or conditions other than age-related macular degeneration and pathologic myopia (Carneiro et al. (2011) Ophthalmologica 225:81-88, Gupta et al. (2010) Eye 24:203-213, Rouvas et al. (2011) Retina 31:871-879, Salehipour et al. (2010) J Ophthalmic Vis Res 5:10-19). However, in many of the studies at least a subgroup of the patients required multiple injections (e.g., more than 3 injections or more than 6 injections) with limited improvement of visual acuity. Since administration of ranibizumab and bevacizumab require intravitreal injections, patient compliance may be reduced, in particular, where multiple injections are needed to delay or halt vision loss.

CNV can lead to permanent vision loss if left untreated. It is an object of the invention to provide further and improved treatments for CNV from causes other than age-related macular degeneration and pathologic myopia.

### DISCLOSURE OF THE INVENTION

The present invention relates to novel treatments of CNV from causes other than age-related macular degeneration and pathologic myopia. In particular, the present invention relates to the use of a non-antibody VEGF antagonist in the treatment of CNV secondary to diseases other than age-related macular degeneration and pathologic myopia. The invention further provides treatment schedules that reduce the total number of doctor visits leading to greater patient compliance and better overall disease outcomes such as stabilization or improvement of visual acuity.

The invention also provides a non-antibody VEGF antagonist for use in a method for treating a patient having CNV secondary to a disease or condition other than age-related macular degeneration and pathologic myopia, wherein said method comprises administering to the eye of a patient a non-antibody VEGF antagonist. The non-antibody VEGF antagonist may be administered intravitreally, e.g. through injection, or topically, e.g. in the form of eye drops.

The invention further provides the use of a non-antibody VEGF antagonist in the manufacture of a medicament for treating a patient having CNV secondary to a disease or condition other than age-related macular degeneration and pathologic myopia.

Non AntibodyVEGF Antagonists

VEGF is a well-characterised signal protein which stimulates angiogenesis. Two antibody VEGF antagonists have been approved for human use, namely ranibizumab (Lucentis®) and bevacizumab (Avastin®). Patients suffering from CNV associated with diseases or conditions other than age-related macular degeneration and pathologic myopia have been treated with bevacizumab and ranibizumab with varied success (Carneiro et al. (2011) Ophthalmologica 225:81-88, Gupta et al. (2010) Eye 24:203-213, Rouvas et al. (2011) Retina 31:871-879, Salehipour et al. (2010) J Ophthalmic Vis Res 5:10-19).

In one aspect of the invention, the non-antibody VEGF antagonist is an immunoadhesin. One such immuoadhesin is aflibercept (Eylea®), which has recently been approved for human use and is also known as VEGF-trap (Holash et al. (2002) PNAS USA 99:11393-98; Riely & Miller (2007) Clin Cancer Res 13:4623-7s). Aflibercept is the preferred non-antibody VEGF antagonist for use with the invention. Aflibercept is a recombinant human soluble VEGF receptor fusion protein consisting of portions of human VEGF receptors 1 and 2 extracellular domains fused to the Fc portion of human IgG1. It is a dimeric glycoprotein with a protein molecular weight of 97 kilodaltons (kDa) and contains glycosylation, constituting an additional 15% of the total molecular mass, resulting in a total molecular weight of 115 kDa. It is conveniently produced as a glycoprotein by expression in recombinant CHO K1 cells. Each monomer can have the following amino acid sequence (SEQ ID NO: 1):

SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

and disulfide bridges can be formed between residues 30-79, 124-185, 246-306 and 352-410 within each monomer, and between residues 211-211 and 214-214 between the monomers.

Another non-antibody VEGF antagonist immunoadhesin currently in pre-clinical development is a recombinant human soluble VEGF receptor fusion protein similar to VEGF-trap containing extracellular ligand-binding domains 3 and 4 from VEGFR2/KDR, and domain 2 from VEGFR1/Flt-1; these domains are fused to a human IgG Fc protein fragment (Li et al., 2011 Molecular Vision 17:797-803). This antagonist binds to isoforms VEGF-A, VEGF-B and VEGF-C. The molecule is prepared using two different production processes resulting in different glycosylation patterns on the final proteins. The two glycoforms are referred to as KH902 (conbercept) and KH906. The fusion protein can have the following amino acid sequence (SEQ ID NO:2):

MVSYWDTGVLLCALLSCLLLTGSSSGGRPFVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPEVAFGSGMESLVEATVGERVR
LPAKYLGYPPPEIKWYKNGIPLESNHTIKAGHVLTIMEVSERDTGNYTVI
LTNPISKEKQSHVVSLVVYVPPGPGDKTHTCPLCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKATP
PVLDSDGSFFLYSKLTVDKSRWQQGNVFSCSVMHEALHNHYTQKSLSLSP
GK

and, like VEGF-trap, can be present as a dimer This fusion protein and related molecules are further characterized in EP1767546.

Other non-antibody VEGF antagonists include antibody mimetics (e.g. Affibody® molecules, affilins, affitins, anticalins, avimers, Kunitz domain peptides, and monobodies) with VEGF antagonist activity. This includes recombinant binding proteins comprising an ankyrin repeat domain that binds VEGF-A and prevents it from binding to VEGFR-2. One example for such a molecule is DARPin® MP0112. The ankyrin binding domain may have the following amino acid sequence (SEQ ID NO: 3):

GSDLGKKLLEAARAGQDDEVRILMANGADVNTADSTGWTPLHLAVPWGHL
EIVEVLLKYGADVNAKDFQGWTPLHLAAAIGHQEIVEVLLKNGADVNAQD
KFGKTAFDISIDNGNEDLAEILQKAA

Recombinant binding proteins comprising an ankyrin repeat domain that binds VEGF-A and prevents it from binding to VEGFR-2 are described in more detail in WO 2010/060748 and WO 2011/135067.

Further specific antibody mimetics with VEGF antagonist activity are the 40 kD pegylated anticalin PRS-050 and the monobody angiocept (CT-322).

The non-antibody VEGF antagonist may be modified to further improve their pharmacokinetic properties or bioavailability. For example, a non-antibody VEGF antagonist may be chemically modified (e.g., pegylated) to extend its in vivo half-life. Alternatively or in addition, it may be modified by glycosylation or the addition of further glycosylation sites not present in the protein sequence of the natural protein from which the VEGF antagonist was derived.

Variants of the above-specified VEGF antagonists that have improved characteristics for the desired application may be produced by the addition or deletion of amino acids. Ordinarily, these amino acid sequence variants will have an amino acid sequence having at least 60% amino acid sequence identity with the amino acid sequences of SEQ ID NO: 1, SEQ ID NO: 2 or SEQ ID NO: 3, preferably at least 80%, more preferably at least 85%, more preferably at least 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with SEQ ID NO: 1, SEQ ID NO: 2 or SEQ ID NO: 3, after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences.

Non-antibody VEGF antagonists are preferred herein over antibody VEGF antagonists due their different pharmacokinetic profile when administered intravitreally. Preferably, the non-antibody VEGF antagonist of the invention binds to VEGF via one or more protein domain(s) that are not derived from the antigen-binding domain of an antibody. The non-antibody VEGF antagonist of the invention are preferably proteinaceous, but may include modifications that are non-proteinaceous (e.g., pegylation, glycosylation).

Patient

In one aspect of the invention, non-antibody VEGF antagonists are particularly useful for treating patients with CNV from causes other than age-related macular degeneration and pathologic myopia.

These causes may include inflammatory processes triggered by an infection with a fungus, a roundworm or protozoan. In other cases, an autoimmune response may be the underlying cause of inflammation. For example, CNV may occur secondarily to uveitis, toxoplasmosis, multifocal chorioditis, (presumed) ocular histoplasmosis, punctate inner choroidopathy, scleroderma, serpiginous choriodopathy, and Vogt-Koyanagi-Harada syndrome.

Other cases of CNV may be idiopathic in origin. For example, CNV is observed in patients having angioid streaks or central serous chorioretinopathy, and there is not always an obvious link to an underlying disease or disorder. CNV can also be associated with rare benign tumours such as choroidal osteoma or certain genetic diseases such as pseudoxanthoma elasticum, Paget's disease, sickle cell anemia and Ehlers-Danlos Syndrome. These diseases may be associated with the formation of angioid streaks, with CNV usually developing secondarily to angioid streaks.

A patient's medical history is usually used to determine the underlying cause for the development of CNV. The medical history as well as previous treatments may inform specific treatment options, in particular for combination treatments. For example, LPT or PDT may not be used in patients who previously responded with CNV to laser therapy. For patients in whom CNV may be triggered by an inflammatory response, combination therapy with an anti-inflammatory agent can be considered. The patient's age, family history and diagnostic testing for the above mentioned diseases can further be used to aid diagnosis of CNV that is secondary to causes other than age-related macular degeneration and pathologic myopia.

Patients with chronic CNV who require multiple intravitreal injections (e.g., more than 3 injections, preferably more than 6 injections) of a VEGF antagonist other than the non-antibody VEGF antagonist of the invention will benefit in particular from the non-antibody therapies of the invention. This includes patients suffering from idiopathic CNV as well as patient with CNV secondary to angioid streaks or central serous chorioretinopathy. Similarly patients suffering from inflammatory CNV, in particular those with Vogt-Koyanagi-Harada syndrome, will generally need more than three injections to see a long lasting benefit from VEGF antagonist treatment. Patients with CNV secondary to a genetic disease such as pseudoxanthoma elasticum also belong to this group.

In many cases, patients will have received prior treatment including therapies other than VEGF antagonist treatment. Laser treatment in some cases can itself lead to CNV, and patients having received several rounds of laser treatment may particularly benefit from the non-antibody VEGF antagonist therapy of the present invention. Similarly, patients who previously have been treated with steroids to reduce the inflammation that may cause CNV may advantageously be treated with a non-antibody VEGF antagonist. The greatest benefit is likely to be seen in patients that have become at least partially refractory to standard treatment with an anti-inflammatory agent.

Administration

The non-antibody VEGF antagonist of the invention will generally be administered to the patient via intravitreal injection, though other routes of administration may be used, such as a slow-release depot, an ocular plug/reservoir or eye drops. Administration in aqueous form is usual, with a typical volume of 20-150 μl e.g. 40-60 μl, or 50 μl. Injection can be via a 30-gauge×½-inch (12.7 mm) needle. For example, aflibercept is generally administered via intravitreal injection at a dose of 2 mg (suspended in 0.05 mL buffer comprising 40 mg/mL in 10 mM sodium phosphate, 40 mM sodium chloride, 0.03% polysorbate 20, and 5% sucrose, pH 6.2). However, the normal dose may be reduced for the treatment of smaller children and in particular infants. The dose for treating an infant with a VEGF antagonist of the invention is usually 50% of the dose administered to an adult. Smaller doses (e.g., 0.5 mg per monthly injection) may also be used.

Alternatively, an intravitreal device is used to continuously deliver a non-antibody VEGF antagonist into the eye over a period of several months before needing to be refilled by injection. Various intravitreal delivery systems are known in the art. These delivery systems may be active or passive. For example, WO 2010/088548 describes a delivery system having a rigid body using passive diffusion to deliver a therapeutic agent. WO 2002/100318 discloses a delivery system having a flexible body that allows active administration via a pressure differential. Alternatively, active delivery can be achieved by implantable miniature pumps. An example for an intravitreal delivery system using a miniature pump to deliver a therapeutic agent is the Ophthalmic MicroPump System™ marketed by Replenish, Inc. which can be programmed to deliver a set amount of a therapeutic agent for a pre-determined number of times.

The non-antibody VEGF antagonist is typically encased in a small capsule-like container (e.g., a silicone elastomer cup).The container is usually implanted in the eye above the iris. The container comprises a release opening. Release of the non-antibody VEGF antagonist may be controlled by a membrane positioned between the non-antibody VEGF antagonist and the opening, or by means of a miniature pump connected to the container. Alternatively, the non-antibody VEGF antagonist may be deposited in a slow-release matrix that prevents rapid diffusion of the antagonist out of the container.

Preferably, the intravitreal device is designed to release the non-antibody VEGF antagonist at an initial rate that is higher in the first month. The release rate slowly decreases, e.g., over the course of the first month after implantation, to a rate that is about 50% less than the initial rate. The container may have a size that is sufficient to hold a supply of the non-antibody VEGF antagonist that lasts for about four to six months. Since a reduced dose of VEGF antagonist may be sufficient for effective treatment when administration is continuous, the supply in the container may last for one year or longer, preferably about two years, more preferably about three years.

Continuous administration via an intravitreal device may be particularly suitable for patients with chronic CNV secondary to, e.g., angioid streaks, central serous chorioretinopathy, Vogt-Koyanagi-Harada syndrome, or pseudoxanthoma elasticum. Patients with CNV refractory to conventional treatment with anti-inflammatory therapy may also be benefit from continuous administration. Because only a small surgery is required to implant a delivery system and intravitreal injections are avoided, patient compliance issues with repeated intravitreal injections can be avoided. Intravitreal concentrations of the non-antibody VEGF antagonist are reduced, and therefore the potential risk of side-effects from non-antibody VEGF antagonist entering the circulation is decreased. This aspect may be of a particular advantage in children who may require general anaesthesia for intravitreal injections. Systemically elevated non-antibody VEGF antagonist levels may interfere with normal growth and development of children who therefore may benefit from lower intravitreal concentrations of the non-antibody VEGF antagonist.

In one aspect of the invention, the non-antibody VEGF antagonist is provided in a pre-filled sterile syringe ready for administration. Preferably, the syringe has low silicone content. More preferably, the syringe is silicone free. The syringe may be made of glass. Using a pre-filled syringe for delivery has the advantage that any contamination of the sterile antagonist solution prior to administration can be avoided. Pre-filled syringes also provide easier handling for the administering ophthalmologist.

Slow-Release Formulations

Non-antibody VEGF antagonists may be provided as slow-release formulations. Slow-release formulations are typically obtained by mixing a therapeutic agent with a biodegradable polymer or encapsulating it into microparticles. By varying the manufacture conditions of polymer-based delivery compositions, the release kinetic properties of the resulting compositions can be modulated.

A slow-release formulation in accordance with the invention typically comprises a non-antibody VEGF antagonist, a polymeric carrier, and a release modifier for modifying a release rate of the non-antibody VEGF antagonist from the polymeric carrier. The polymeric carrier usually comprises one or more biodegradable polymers or co-polymers or combinations thereof. For example, the polymeric carrier may be selected from poly-lactic acid (PLA), poly-glycolic acid (PGA), poly-lactide-co-glycolide (PLGA), polyesters, poly (orthoester), poly(phosphazine), poly (phosphate ester), polycaprolactones, or a combination thereof. A preferred polymeric carrier is PLGA. The release modifier is typically a long chain fatty alcohol, preferably comprising from 10 to 40 carbon atoms. Commonly used release modifiers include capryl alcohol, pelargonic alcohol, capric alcohol, lauryl alcohol, myristyl alcohol, cetyl alcohol, palmitoleyl alcohol, stearyl alcohol, isostearyl alcohol, elaidyl alcohol, oeyl alcohol, linoleyl alcohol, polyunsaturated elaidolinoleyl alcohol, polyunsaturated linolenyl alcohol, elaidolinolenyl alcohol, polyunsaturated ricinoleyl alcohol, arachidyl alcohol, behenyl alcohol, erucyl alcohol, lignoceryl alcohol, ceryl alcohol, montanyl alcohol, cluytyl alcohol, myricyl alcohol, melissyl alcohol, and geddyl alcohol.

Preferably, the non-antibody VEGF antagonist is incorporated into a microsphere-based sustained release composition. The microspheres are preferably prepared from PLGA. The amount of non-antibody VEGF antagonist incorporated in the microspheres and the release rate of the non-antibody VEGF antagonist can be controlled by varying the conditions used for preparing the microspheres. Processes for producing such slow-release formulations are described in U.S. 2005/0281861 and U.S. 2008/0107694.

Treatment Regimens

Non-antibody VEGF antagonists of the invention allow increased spacing between administrations resulting in a more cost-effective therapy. In addition, better patient compliance is achieved when intravitreal injections have to be performed less frequently. This is particularly advantageous in patients suffering from CNV secondary to diseases or conditions other than age-related macular degeneration and pathologic myopia who may require multiple injections to improve visual acuity or prevent vision loss.

In some cases, a single injection of the VEGF antagonist according to the invention may be sufficient to ameliorate the disease or prevent disease progression for many years. In other cases, three injections each one month apart are administered to the patient, while any subsequent injections are performed less frequently. In certain cases, two injections spaced 6 weeks apart, preferably 8 weeks apart, more preferably 10 weeks apart may be required to improve visual acuity or halt disease progression. In other cases, three or more injections may be needed. In these cases, three injections each one month apart may be administered to the patient, while any subsequent injections are performed less frequently or on an as needed-basis. For example, for any subsequent injections after the first three injections, the time between injections may be at least 6 weeks, preferably 8 weeks, more preferably 10 weeks apart. Treatment may be continued until maximum visual acuity is achieved. For example, treatment may be discontinued when visual acuity is stable for at least three months (i.e., no increase or decrease in visual acuity is observed during this period).

Disease progression or recurrence of CNV may require one or more or continued treatment cycles. For example, in a first cycle, two or more injections spaced 4 weeks, 6 weeks, preferably 8 weeks, more preferably 10 weeks apart may be administered followed by an interruption of treatment for 3 months, 4 months, 5 months, 6 months, 9 months, 12 months, 24 months or 36 months. If CNV reappears, the treatment is continued with a second cycle. In some cases three, four, five or more treatment cycles may be needed. For examples, a further treatment cycle may be initiated if worsening of visual acuity is observed (e.g., by monthly checking a patient's vision after treatment has been discontinued). In one embodiment, the treatment may comprise two or more (preferably 3) injections spaced 4 weeks apart, followed by two or more injections spaced 8 weeks apart.

In another aspect of the invention, the non-antibody VEGF antagonist according to the invention is administered as needed. The non-antibody VEGF antagonist is administered the first time after an initial diagnosis of CNV has been made. A diagnosis of CNV can be made during examination of the eye by a combination of slit-lamp evaluation and biomicroscopic fundus examination with optical coherence tomography (OCT) and/or fluorescein fundus angiography. A second, third or further administration of the non-antibody VEGF antagonist is performed only if examination of the eye reveals signs of persistent or recurring CNV. Alternatively, three injections each one month apart are administered to the patient after the initial diagnosis of CNV, while any subsequent injections are performed on an as needed-basis.

Combination Therapy

The compounds of the invention may be used in combination with one or more additional treatment.

In one aspect of the invention, treatment with a VEGF antagonist of the invention may be used in combination with LPT or PDT.

LPT uses laser light to cause controlled damage of the retina to produce a beneficial therapeutic effect. Small bursts of laser light can seal leaky blood vessels, destroy abnormal blood vessels, seal retinal tears, or destroy abnormal tissue in the back of the eye. It is quick, a non-invasive thermal photocoagulation, and usually requires no anaesthesia other than an anaesthetic eye drop. LPT techniques and apparatuses are readily available to ophthalmologists. See Lock et al. (2010) Med J Malaysia 65:88-94

LPT techniques can be classified as focal, panretinal (or scatter), or grid. Focal LPT applies small-sized burns to specific areas of focal leakage (microaneurysms) in the macula. Panretinal LPT scatters burns throughout the peripheral retina. Grid LPT applies a pattern of burns to macular areas with diffuse capillary leakage or non-perfusion, with each burn typically spaced apart by two visible burn widths. Patients can receive more than one type of LPT (e.g. a combination of focal and panretinal LPT) and these may be administered one directly after the other, or after a delay. A typically therapeutic panretinal LPT involves the application of 1200-1600 burns.

Laser spot sizes (spot diameters) of 50-500 μm are typical (smaller spot sizes are more usual for focal LPT, larger for panretinal), applied for 50-200 ms (continuously, or via micropulses), using green-to-yellow wavelengths e.g. using an argon gas (514.5 nm) laser, a frequency-doubled Nd-YAG (532 nm) laser, a krypton yellow laser (568 2 nm), or a tunable dye laser (variable wavelength). In some cases a red laser may be used if a green or yellow laser is precluded (e.g. if vitreous hemorrhage is present).

Micropulse laser therapy (MLP) uses 810 nm or 577 nm lasers to direct a discontinuous beam of laser light on the affected tissue (Kiire et al. (2011) Retina Today, 67-70). This results in a greater degree of control over the photothermal effects in laser photocoagulation. The steady continuous-wave emission of conventional LPT is delivered in form of short laser pulses. Each pulse typically is 100-300 μs in length with a 1700 to 1900 82 s interval between each pulse. The “width” (“ON” time) of each pulse and the interval between pulses (“OFF” time) are adjustable by the surgeon. A shorter micropulse “width” limits the time for the laser-induced heat to spread to adjacent tissue. A longer interval between pulses allows cooling to take place before the next pulse is delivered. Intraretinal damage thus can be avoided. Hence MLP is also referred to as “sub-threshold laser treatment” or “tissue-sparing laser therapy”. 10-25% of micropulse power is sufficient to show a consistent photothermal effect that is confined to the retinal pigment epithelium and does not affect the neurosensory retina.

According to the invention, patients can receive both LPT and a non-antibody VEGF antagonist. Administration of LPT and the antagonist should not occur simultaneously, so one will precede the other. The initiation of LPT and of antagonist administration occur within 6 months of each other, and ideally occur within 1 month of each other (e.g. within 10 days).

Typically, antagonist therapy is administered prior to LPT. LPT can take place promptly after antagonist administration (e.g. within 2-20 days, typically within 3-10 days), or can take place after a longer delay (e.g. after at least 4 weeks, after at least 8 weeks, after at least 12 weeks, or after at least 24 weeks). Injected antagonists are expected to maintain significant intravitreal VEGF-binding activity for 10-12 weeks (Stewart & Rosenfeld (2008) Br J Ophthalmol 92:667-8). In an alternative embodiment, the antagonist therapy is administered after LPT.

Some embodiments involve more than one administration of LPT and/or of antagonist. For instance, in one useful embodiment a patient receives in series (i) antagonist (ii) at least one administration of LPT (iii) antagonist. For instance, the patient may receive an initial intravitreal injection of antagonist; then, within 10-14 days of receiving the antagonist, they receive focal LPT; followed by a second injection of the antagonist 4 weeks or a month after the initial injection. Alternatively, within 10-14 days of receiving a VEGF antagonist, a patient may receive at least one sitting (e.g. up to three) of panretinal LPT; and then, 4 weeks or a month after the initial injection, the patient receives a second injection of the VEGF antagonist. This regimen may be continued with further doses of the antagonist e.g. with a frequency of every 1 or 2 months. By ensuring that the photocoagulation is initiated within 14 days of the initial injection, the antagonist will still be present in the eye.

PDT uses a light-activated molecule to cause localised damage to neovascular endothelium, resulting in vessel occlusion. Light is delivered to the retina as a single circular spot via a fiber optic cable and a slit lamp, using a suitable ophthalmic magnification lens (“cold” laser light application/treatment). The light-activated compound is injected into the circulation prior to the laser light application, and damage is inflicted by photoactivation of the compound in the area afflicted by CNV. One commonly used light-activated compound is verteporfin (Visudyne®). Verteporfin is transported in the plasma primarily by lipoproteins. Once verteporfin is activated by light in the presence of oxygen, highly reactive, short-lived singlet oxygen and reactive oxygen radicals are generated which damages the endothelium surrounding blood vessels. Damaged endothelium is known to release procoagulant and vasoactive factors through the lipo-oxygenase (leukotriene) and cyclooxygenase (eicosanoids such as thromboxane) pathways, resulting in platelet aggregation, fibrin clot formation and vasoconstriction. Verteporfin appears to somewhat preferentially accumulate in neovasculature. The wavelength of the laser used for photoactivation of the light-activated compound may vary depending on the specific light-activated compound used. For example, 689 nm wavelength laser light delivery to the patient 15 minutes after the start of the 10-minute infusion with verteporfin may be used. Photoactivation is controlled by the total light dose delivered. Using verteporfin in the treatment of CNV by PDT, the recommended light dose is 50 J/cm² of neovascular lesion administered at an intensity of 600 mW/cm² over 83 seconds. Light dose, light intensity, ophthalmic lens magnification factor and zoom lens setting are important parameters for the appropriate delivery of light to the predetermined treatment spot during PDT and may need to be adapted depending on the laser system used for therapy.

Administration of the non-antibody VEGF antagonist is performed before or after photodynamic therapy. Typically, administration of the non-antibody VEGF antagonist and PDT will be performed on the same day (e.g. within 24 hours of one another). If administration of the non-antibody VEGF antagonist and PDT are performed on the same day, typically the PDT will be administered first. In one embodiment, treatment with non-antibody antagonist is started up to 48 hours before photodynamic therapy. Alternatively, treatment with the non-antibody VEGF antagonist is initiated at least 1 week, 2, weeks, 3 weeks, 4 weeks, 2 months, 3 months, 4 months, 5 months or 6 months before PDT. The non-antibody VEGF antagonist may be administered every 4 weeks, every 6 weeks, or every 8 weeks, or may be administered as needed. Where the non-VEGF antagonist is administered at specified intervals, treatment may be continued at the same intervals or extended intervals after PDT. Where the interval is extended, the period between administration of the non-antibody VEGF antagonist may increase by 50% or 100%. For example, if the initial interval was 4 weeks, the interval may be extended to 6 or 8 weeks. Alternatively, non-antibody VEGF antagonist administration may be continuous, for example, if an intravitreal delivery system is used. The intravitreal device may be implanted prior to PDT. Alternatively, a single administration of non-antibody VEGF antagonist shortly before or after PDT may be sufficient to achieve the desired effect. For example, a single dose of non-antibody VEGF antagonist may be given on the day of the PDT.

PDT may be repeated as needed. Generally, PDT with verteporfin it is not given more frequently than every 3 months, and may be repeated every 3 months. For example, treatment may be continued until there has been complete regression of CNV leakage in the treated eye(s). Alternatively, PDT may be repeated less frequently, in particular if the non-antibody VEGF antagonist treatment is continued after PDT. For example, intervals between PDT may be extended to every 4 months, every 5 months, or every 6 months. Ideally, continued treatment with a non-antibody VEGF antagonist after PDT prevents recurrence of CNV.

In a further aspect of the invention, treatment time and patient compliance is improved by using a non-antibody VEGF antagonist in combination with an anti-inflammatory agent. Administering the VEGF antagonist in combination with an anti-inflammatory agent can have synergistic effects depending on the underlying cause of CNV. Addition of an anti-inflammatory agent is particularly advantageous in CNV secondary to an inflammatory disease or condition. Anti-inflammatory agents include steroids and NSAIDs. NSAIDs used in the treatment of ocular diseases include ketorolac, nepafenac and diclofenac. In some instances, the use of diclofenac is preferred. Corticosteroids used in treating ocular diseases include dexamethasone, prednisolone, fluorometholone and fluocinolone. Other steroids or derivatives thereof that may be used in combination with VEGF antagonist treatment include anecortave, which has angiostatic effects but acts by a different mechanism than the VEGF antagonists according to the invention. A preferred anti-inflammatory agent is triamcinolone. The anti-inflammatory agent may also be a TNF-α antagonist. For example, a TNF-α antibody may be administered in combination with a non-antibody VEGF antagonist. TNF-α antibodies, e.g. those sold under the trade names Humira®, Remicade®, Simponi® and Cimzia®, are well known in the art. Alternatively, a TNF-α non-antibody antagonist such as Enbrel® may be administered in combination with a non-antibody VEGF antagonist.

The anti-inflammatory agent may be administered at the same time as the non-antibody VEGF antagonist. The anti-inflammatory agent can be administered either systemically or locally. For example, the anti-inflammatory agent may be administered orally, topically, or, preferably, intravitreally. In a preferred embodiment, triamcinolone is administered intravitreally at the same time as the non-antibody VEGF antagonist of the invention.

In yet another aspect of the invention, the non-antibody VEGF antagonist is administered after administration of an antimicrobial agent. For example, the antimicrobial agent may be selected from gatifloxacin, ciprofloxacin, ofloxacin, norfloxacin, polymixin B+chloramphenicol, chloramphenicol, gentamicin, fluconazole, sulfacetamide, tobramycin, neomycin+polymixin B, and netilmicin. Alternatively, the antimicrobial agent may be selected from pyrimethamine, sulfadiazine and folinic acid or a combination thereof. Combination with pyrimethamine can be particularly advantageous in treating patient with CNV associated with toxoplasmosis.

General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

### MODES FOR CARRYING OUT THE INVENTION

### COMPARATIVE EXAMPLE

Twenty-one eyes of 20 patients with CNV secondary to diseases other than age-related macular degeneration and pathologic myopia were enrolled in a clinical study. Mean age of the patients at enrolment was 46.5 years (range 10-80). 13 CNV lesions were subfoveal, 5 juxtafoveal and 3 extrafoveal. Nine eyes had angioid streaks, 5 inflammatory chorioretinal diseases, 3 presented with a previous diagnosis of central serous chorioretinopathy, and the remaining 4 eyes had idiopathic CNV. Six patients moved to ranibizumab treatment after previous PDT treatment. The remaining patients received ranibizumab as primary therapy. All patients had evidence of fluorescein angiography leakage at baseline.

Visual acuity measurements were performed at the first visit with Early Treatment Diabetic Retinopathy Study (ETDRS) charts, and best-corrected visual acuity (BCVA) was obtained. The mean initial BCVA ETDRS score was 54.5±15.7 letters (20/80). Intravitreal injection of 0.5 mg ranibizumab was carried out using a 30-gauge needle. Patients were given topical ofloxacin 4 times daily, 3 days before and 3 days after each injection.

Patients were re-evaluated every 4 weeks after the first injection of ranibizumab. Re-evaluation included the presence of metamorphopsia, BCVA, slit-lamp evaluation, biomicroscopic fundus examination, ocular coherence tomography (OCT) and/or fluorescein angiography. Ocular imaging consisted of fluorescein angiography (performed at baseline in all cases) and/or OCT at each follow-up visit. Treatment was continued if intraretinal or subretinal fluid, subfoveal or juxtafoveal leakage and/or metamorphopsia were detected during a follow-up visit.

Twenty-one eyes completed 90 days of follow-up, and 16 (76%) completed 180 days. Mean total follow-up of 206 days (range 90-723). Overall, BCVA increased +9.8 letters with treatment (p=0.015). The mean gain after the first injection of intravitreal ranibizumab was +5.8 letters (p=0.01). The improvement at 90 days was +10.4 (p=0.001) and at 180 days +11.1 ETDRS letters (p=0.002). A trend for better visual acuity results was observed as the time of follow-up increased until the 6-month evaluation, with a slow decrease thereafter. Sixty per cent of eyes increased their ETDRS score, with a visual gain ≧15 letters occurring in 9 eyes (43%). Visual loss ≧15 letters occurred in 2 eyes with angioid streaks and subfoveal CNV. One patient developed subretinal fibrosis and the second eye received 11 injections without resolution of retinal edema. The overall mean number of injections per eye was 3.57 (range 1-11). Mean macular thickness decreased from 306.43±150.80 to 264.14±138.65 μm after the initial injection (p=0.09). The mean macular thickness decreased significantly at 90 (p=0.03) and 180 (p=0.04) days when compared to baseline, with an overall decrease of −56.81 μm (p=0.049). No systemic or ocular complications were observed during the study. None of the eyes received concurrent local therapy during follow-up.

### EXAMPLE 1

Five patients are enrolled in an open-label, single-arm study to assess the efficacy and safety of intravitreal injections of aflibercept for the treatment of CNV secondary to presumed ocular histoplasmosis syndrome.

Patents receive intravitreal injections of aflibercept every 4 weeks (monthly) for the first 3 months followed by intravitreal injection every eight weeks (two months) for up to 12 months. Aflibercept is dosed at 2.0 mg (0.05 mL) per injection. Dosing at monthly intervals is allowed if needed in the opinion of the investigator based on presence of fluid on OCT and/or a decrease in visual acuity of greater than or equal to five letters from the previous visit.

The primary endpoint of the study will be an assessment of incidence and severity of adverse events over a 12-month treatment period. Secondary outcome measures are (i) mean visual acuity (BCVA) at months 6 and 12; (ii) mean change in central foveal thickness as measured by OCT from baseline at months 6 and 12; (iii) mean change in macular volume from baseline at months 6 and 12; (iv) mean change in BCVA from baseline at months 6 and 12; (v) mean change in CNV lesion characteristics (size, leakage, etc.) from baseline at months 6 and 12; and (vi) the proportion of patients with no fluid on OCT (absence of cystic edema and subretinal fluid) at months 6 and 12.

### EXAMPLE 2

This study monitors safety outcomes for patients being treated with intravitreal aflibercept injections for choroidal neovascularization (CNV) secondary to presumed ocular histoplasmosis syndrome.

Male and female patients, 18 years and older, are enrolled in an open-label, two-arm study to assess the efficacy and safety of intravitreal injections of aflibercept for the treatment of CNV secondary to presumed ocular histoplasmosis syndrome. Patients who are diagnosed with active CNV secondary to presumed ocular histoplasmosis demonstrated by active leakage on fluorescein angiography with spectal domain OCT evidence of subretinal or intraretinal fluid or PED, are included in the study. Active CNV may also be defined as demonstrating active subretinal haemorrhage. In addition, patients are suitable for inclusion, who have ETDRS best corrected visual acuity of 20/20-20/320.

Patients are excluded who (i) are under 18 years of age; (ii) have CNV due to causes other than presumed ocular histoplasmosis; (iii) have undergone previous treatment in the study eye within 6 months prior to Day 1; (iv) have had more than 5 intravitreal injections of anti-VEGF therapy within the previous 12 months; (v) have any clinical evidence of any ocular condition other than ocular histoplasmosis; (vi) have a history of allergy to fluorescein; (vii) are, in the case of females, pregnant (or planning on becoming pregnant within the next 13 months) or breast feeding women; (viii) are sexually active men or women, and who are not willing to practice more than one form of contraceptive during the next 13 months; (ix) have undergone previous (within previous 3 months) systemic anti-VEGF therapy.

Patients are randomized to two treatment groups: group A or group B. Group A patients receive intravitreal aflibercept injections monthly for 3 months (Baseline, Months 1 and 2), followed by intravitreal aflibercept injection every 2 months (Months 4, 6, 8 and 10) for 12 months. Monthly visits with evaluations for as-needed intravitreal aflibercept injection are conducted. Group B patients receive one intravitreal aflibercept injection at baseline, followed by monthly visits with evaluations for as-needed dosing of intravitreal aflibercept injection for 12 months.

The primary outcome is the rate of incidents and severity of ocular and systemic adverse events through month 12. Secondary outcome measures are (i) mean change in best corrected visual acuity

(BCVA) from baseline; (ii) proportion of patients gaining more than 5, 10 and 15 letters; (iii) proportion of patients losing more than 5, 10 and 15 letters. Other outcomes are OCT changes, and mean change from baseline in central subfield thickness over time up to 12 months assessed on OCT.

### EXAMPLE 3

This study shows that inflammatory CNV with suboptimal anatomical response to intravitreal ranibizumab therapy and persistent fluid throughout a long-term follow-up benefits from switching to intravitreal aflibercept.

Case 1

A 30-year-old myopic woman diagnosed of multifocal choroiditis (MFC) with secondary CNV in her left eye received 12 intravitreal injections of ranibizumab through a follow-up of 732 days. The patient's mean visual acuity (VA) was 0.96, the mean central subfield thickness (CST) measured 284.5 microns, and the mean interval between injections was 66.5 days. As a consequence of suboptimal anatomical response with persistent intraretinal fluid, therapy was switched to aflibercept.

Patient received four injections of aflibercept through a follow-up of 168 days. The patient's mean VA improved to 1.0 (p=0.317), the mean CST decreased to 266.33 microns (p=0.108), and the mean interval between injections was 56 days (p=0.109).

Case 2

A 57-year-old myopic woman diagnosed with MFC with secondary CNV in her left eye had received six injections of ranibizumab through a follow-up of 286 days. The patient's mean VA was 0.81, the mean CST measured 295 microns, and the mean interval between injections was 57.2 days. As a consequence of suboptimal anatomical response with persistent intraretinal fluid, intravitreal therapy was switched to aflibercept. The patient received three injections of aflibercept through a follow-up of 123 days. The patient's mean VA improved to 0.91 (p=0.399), the mean CST decreased to 287.83 microns (p=0.730), and the mean interval between injections was 60.3 days (p=0.999).

Case 3

A 21-year-old myopic woman diagnosed of MFC with secondary CNV in her left eye had received three intravitreal injections of ranibizumab through a follow-up of 76 days. The patient's mean VA was 0.33, the mean CST measured 233 microns, and the mean interval between injections was 39.3 days. As a consequence of persistent intraretinal fluid, therapy was switched to aflibercept. The patient received three injections of aflibercept through a follow-up of 86 days. The patient's mean VA improved to 0.45 (p=0.144), the mean CST decreased to 201.5 microns (p=0.068), and the mean interval between injections was 47 days (p=0.285).

Thus, there was a general trend for improvement in VA and decrease in CST.

It will be understood that the invention is described above by way of example only and modifications may be made whilst remaining within the scope and spirit of the invention.

1. A method for treating choroidal neovascularisation (CNV) secondary to a disease or condition other than age-related macular degeneration or pathologic myopia in a patient having a retinal disorder, comprising administering to the patient a therapeutically effective amount of a non-antibody VEGF antagonist.

2. (canceled)

3. The method of claim 1, wherein CNV is secondary to an inflammatory condition.

4. The method of claim 3, wherein the inflammatory condition is triggered by an infectious agent or an autoimmune response.

5. The method of claim 1, wherein CNV is secondary to toxoplasmosis, multifocal chorioditis, ocular histoplasmosis, punctate inner choroidopathy, scleroderma, serpiginous choriodopathy, or Vogt-Koyanagi-Harada syndrome.

6. The method of claim 1, wherein CNV is secondary to a tumour.

7. The method of claim 1, wherein CNV is secondary to a genetic disease.

8. The method of claim 1, wherein CNV is secondary to angioid streaks or central serous chorioretinopathy.

9. The method of claim 1, wherein the non-antibody antagonist is selected from a recombinant human soluble VEGF receptor fusion protein and a recombinant binding protein comprising an ankyrin repeat domain that binds VEGF-A.

10. The method of claim 1, wherein the patient has received more than three injections of a VEGF antagonist other than the non-antibody VEGF antagonist of claim 9.

11. The method of claim 1, wherein the non-antibody VEGF antagonist is aflibercept.

12. The method of claim 11, wherein aflibercept is administered via intravitreal injection.

13. The method of claim 11, wherein the aflibercept is administered at a dose of 2 mg.

14. The method of claim 1, wherein the patient suffers from CNV refractory to conventional treatment.

15. The method of claim 14, wherein the patient suffers from CNV refractory to treatment with ranibizumab or bevacizumab.

16. The method of claim 1, wherein the method further comprises administering an anti-inflammatory agent.

17. The method of claim 12, wherein both the non-antibody VEGF antagonist and the anti-inflammatory compound are administered intravitreally.

18. The method of claim 1, wherein the anti-inflammatory agent is administered at the same time as the non-antibody VEGF antagonist.

19. The method of claim 1, wherein the non-antibody VEGF antagonist is administered every 4 weeks, every 6 weeks, every 8 weeks or every 10 weeks.

20. The method of claim 19, wherein the non-antibody VEGF antagonist is administered every 4 weeks.

21. The method of claim 1, wherein the non-antibody VEGF antagonist is administered two or more times, preferably three times, every 4 weeks, followed by two or more administrations every 8 weeks.

22. The method of claim 1, wherein the non-antibody VEGF antagonist is administered continuously.

23. The method of claim 1, wherein a first dose of the non-antibody VEGF antagonist is administered after the initial diagnosis of CNV and wherein a second dose of the non-antibody VEGF antagonist is administered only if CNV persists or recurs after administration of the first dose.

24. The method of claim 23, wherein the interval between the first and the second treatment is at least 4 weeks, at least 6 weeks, at least 8 weeks or at least 10 weeks.

25. The method of claim 23, wherein the interval between the first and the second treatment is at least 3 months, 6 month or 9 months.
